(19) United States
(12) Patent Application Publication
(10) Pub. No.: US 2005/0064424 A1
(43) Pub. Date: Mar. 24, 2005

(54) Method of identifying novel proteins

(76) Inventor: Grace Wong, Brookline, MA (US)

Correspondence Address: David S Resnick;Nixon Peabody, 100 Summer Street, Boston, MA, 02110-2131, US

(21) Appl. No.: 10/500,267

(22) PCT Filed: Dec. 19, 2002
(86) PCT No.: PCT/US02/40881

Related U.S. Application Data
(60) Provisional application No. 60/344,293, filed on Dec. 21, 2001.

(51) Int. Cl. C12Q 1/68; C12N 15/85

(52) U.S. Cl. 435/6; 435/455

(57) ABSTRACT

The present invention relates to a method of identifying a novel protein by exposing, in vivo or in vitro, a cell source or a mixture of cell sources in culture to one or more stimulatory factors. The invention is based on the discovery that exposure of cells to stimulatory factors results in expression of novel proteins, including secreted proteins and intracellular proteins and that the exposed cells can be used to easily identify large numbers of rare transcripts encoding novel proteins.

### BACKGROUND OF THE INVENTION

Expression of proteins by cells is a highly regulated process and only a fraction of the existing genes are constantly expressed in every cell, these genes are generally called household genes. The rest of the genes are expressed as a response of myriad of external stimuli or stress, including paracrine, autocrine and endocrine stimuli, such as hormones, cytokines, temperature, oxygen concentration, pressure and pathogens.

Cytokines are a diverse group of soluble proteins and peptides which act as humoral regulators at nano- to picomolar concentrations and they modulate the functional activities of individual cells and tissues. These proteins also mediate interactions between cells directly and regulate processes taking place in the extracellular environment. In general cytokines act on a wider spectrum of target cells than, e.g., hormones and unlike for hormones, there is not a single organ source for cytokines. The fact that cytokines are secreted proteins also means that the sites of their expression does not necessarily predict the sites at which they exert their biological function. COPE: Cytokines Online Pathfinder Ecyclopaedia, Horst Ibelgauft's Hypertext Information Universe of Cytokines at URL address http://www.copewithcytokines.de/cope.

Cytokine expression is regulated by a myriad of factors as they are important mediators involved in embryogenesis and organ development, such as angiogenesis and neuroimmunological, neuroendocrinological, and neuroregulatory processes. Cytokines are also important positive or negative regulators of mitosis, differentiation, migration, cell survival and cell death as well as transformation. It has also been shown that a number of viral infectious agents exploit the cytokine repertoire of organisms to evade immune responses of the host. COPE: Cytokines Online Pathfinder Ecyclopaedia, Horst Ibelgauft's Hypertext Information Universe of Cytokines at URL address http://www.copewithcytokines.de/cope.

Although a large number of genes for cytokines are already known, it is very likely that the genome still harbors unknown transcript encoding cytokines that would be important targets for drug development. However, systemic identification of novel cytokines is difficult because their primary sequences are rarely closely related, although some appear to have some common three-dimensional features. In addition, these proteins are often not expressed or expressed only at very low levels in cells that are unexposed to specific stimuli.

Currently available methods to identify novel proteins include sequence homology searches that could potentially identify novel cytokines from the existing sequence databases. However, sequence homology generally needs to be rather high for this procedure to be successful. Also, genomic sequencing or sequence comparison to gene databases containing genomic sequences alone does directly reveal the protein encoding sequence because of the interrupting intron structures. In addition, cytokines may be homologous but posses a different function and respond to different stimuli. Also cytokines do not generally share a lot of homology and therefore most of them would be missed in a sequence homology search. Additionally, some homologous cytokines exhibit different functions and responses to different stimuli despite sequence homology. In addition, homology searches do not identify novel proteins; they only identify proteins already defined by nucleotide or amino acid sequence and present in the database. Another approach is to use hybridization techniques using nucleotide probes to search expression libraries for novel proteins. Also this method would have limited applicability to finding novel cytokines due to the low sequence homology and variability in the functional domains.

A number of methods to identify novel proteins are based on functional genomics. These methods include, for example, isolating partner proteins involved in protein-protein interactions, such as yeast two-hybrid system, or assays utilizing known or orphan receptors or antibodies to “fish out” novel proteins. However, also these approaches would not be useful in systemic search of novel cytokines with unknown receptors.

Expression profiling techniques are used to identify transcripts that are exclusively expressed in certain tissues or during development or in disease states (Armen et al. Chapter 2 in Functional Genomics, eds. S. P. Hunt and F. J. Livesey, Oxford University Press., 2000). However, because cytokines are usually expressed only transiently in a variety of tissues and most of the time they are expressed at very low levels, a systemic screen for novel cytokines using these methods alone would not necessarily allow identification sparsely and temporarily expressed cytokine transcripts whose transcription is tightly regulated by external stimuli.

Therefore, a method that is independent from sequence homology, protein-protein interactions, and provides sufficiently high transcript levels of cytokines for detection would be useful in systemic identification of novel cytokines.

### SUMMARY OF THE INVENTION

The present invention is based on the discovery that exposure of cells to stimulatory factors results in expression of novel proteins, including secreted proteins and intracellular proteins. The exposed cells can be used to easily identify large numbers of rare transcripts encoding novel proteins. Nucleic acids isolated from the stimulated cells can thereafter be used to create nucleic acid libraries which, using hybridization-based methods, are reduced to contain nucleic acids the expression of which was stimulated by such stimulatory factors. These nucleic acids form a basis for a novel microarray containing nucleic acids encoding novel proteins.

In one embodiment, the invention provides a method of identifying a novel protein by exposing, in vivo or in vitro, a cell source or a mixture of cell sources in culture to one or more stimulatory factors. A first library of nucleic acids is created from the stimulated cell. A second library of nucleic acids is created from the same cell source or a mixture of cell sources that is not exposed to the stimulatory factors. The nucleic acids of the first and second libraries are then subjected to subtractive hybridization and the remaining nucleic acids are used to create a nucleic acid array. The nucleic acid array is consequently hybridized with a first set of nucleic acids isolated from an other stimulated cell source and a second set of nucleic acids isolated from an unstimulated cell. The hybridization signals on the nucleic acid array that are at least about two times stronger after the hybridization of the frst set compared to the hybridization of the second set are selected. The clones corresponding to the spot on the nucleic acid array are picked from the original library of first set of nucleic acids and subjected to sequencing, preferably partial sequencing. The nucleic acid sequencing is performed from either 5′ or 3′ ends or both ends of the clones and the sequence is subjected to sequence comparison software, e.g. BLAST. If the sequence is less than about 50% homologous with any known sequence in the databases, it is considered a novel sequence. Sequences identified as having a nucleic acid sequence encoding a signal peptide are considered to encode novel secreted proteins. If the clone is not a full length clone, the full length clone can be obtained from any nucleic acid library containing nucleic acids from the organism corresponding to the cell source. The full length clones can be consequently expressed to identify novel secreted proteins.

Alternatively, the proteins can also be expressed and thereafter used to produce antibodies.

The cell source can be any cell type including, but not limited to, epithelial, endothelial, neuronal, adipose, and reproductive cell, such as cumulus, ovarian or sertoli cell. The cell source can be obtained from organs including, but not limited to brain, liver, lung, gut, stomach, fat, muscle, endocrine organs, testes, uterus, cumulus, ovary, skin and bone, etc. of an organism, preferably mammalian organism and most preferably a murine or a human organism that has been administered or subjected to the stimulatory factor.

The stimulatory factors include any stress stimuli, such as hormones, growth factors, cAMP inducers such as forskolin, Ca++ flux inducing molecules such as macrophage-derived chemokine, and other small organic or inorganic molecules or peptides, heat, pressure, radiation, genetic alterations and pathogens, such as bacteria, fungi and viruses. The preferred stimulatory factors include, but are not limited to FSH, LH, TNF, IFNγ, PMA, LPS, cycloheximide, and Indomethacin and combinations thereof. Preferably a mixture of more than one stimulatory factor is used. Most preferably a mixture of FSH, LH, TNF, IFNγ, PMA, LPS, cycloheximide and Indomethacin is used.

In a preferred embodiment, the protein is a secreted protein or intracellular protein, most preferably it is a cytokine.

In another embodiment, the method further includes steps of cloning and sequencing the nucleic acid encoding the novel secreted protein.

The expressed proteins can further be used to create a stimulated protein-specific protein microarray, e.g., cytokine protein array, representing proteins from a cell or tissue that are expressed under stimulatory conditions. The protein microarray can be used to, for example, identify receptors to the proteins.

Moreover, the expressed peptides or proteins can be used to produce antibodies against the proteins.

Additionally, the expressed peptides or proteins can be used to screen a library of peptides, small molecules or antibodies for molecules that interact with the novel proteins.

### BRIEF DESCRIPTION OF THE FIGURES

FIGS. 1A and 1B show a schematic presentation of the creation of activated cDNA libraries. FIG. 1A shows creation of a cDNA library from resting cells. FIG. 1B shows creation of a cDNA library from stimulatory factor activated cells.

FIG. 2 shows 10 novel secreted clones from activated and one control cDNA libraries after EcoRI and Not-1 restriction enzyme digest.

FIGS. 3A and 3B show an analysis of a microarray prepared from total RNA isolated from mice reproductive organs after intraperiotnal in vivo administration of a mixture of stimulatory factors to the mice. The expressed transcripts that were stimulated are circled in FIG. 3A. FIG. 3B illustrates an example of the steps of the present invention.

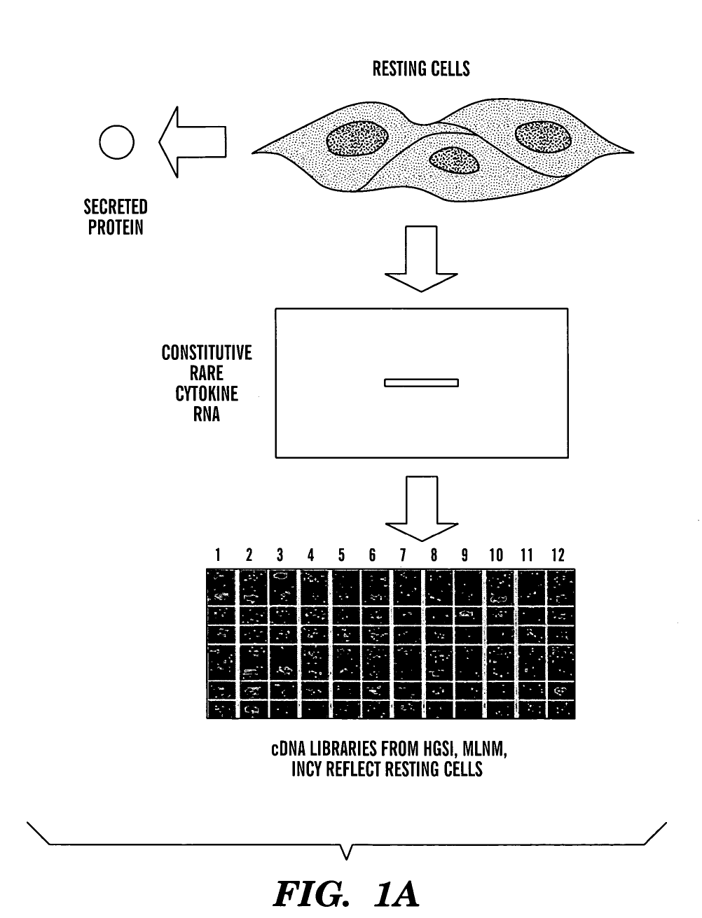

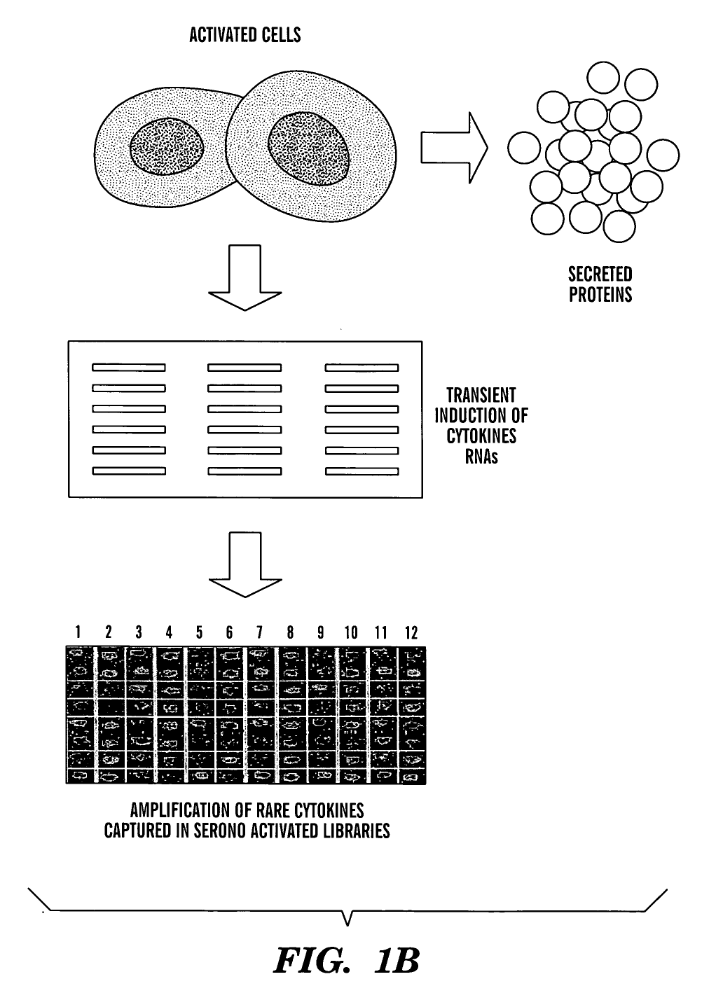

It is to be understood that both the foregoing general description and the following detailed description are merely exemplary of the invention, and are intended to provide an overview or framework for understanding the nature and character of the invention as it is claimed.

### DETAILED DESCRIPTION OF THE INVENTION

The present invention is based upon a discovery that novel proteins, including secreted and intracellular proteins, can be isolated from cells or tissues or organs that are exposed to one or more stimulatory factors. The method allows comparison of same cell or tissue or organ type under normal, quiet, resting or healthy stage and under activated, induced, stimulated or diseased stage after exposure of the cell or tissue to one or more stress or stimulatory factors. The method allows rapid throughput identification of rare and temporarily expressed proteins whose regulation is normally under tight internal and external control. The method also allows identification of functional characteristics as well as interacting molecules of the secreted and intracellular proteins as well as production of antibodies to such novel proteins.

In one embodiment, the invention provides a method of identifying a novel secreted protein by exposing a cell source or a mixture of cell sources in culture or in live organism to one or more stimulatory or stress factors. The terms “activating factors”, “inducing factors”, “stess factors” and “stimulatory factors” are herein used interchangeably and are meant to include all stimuli that can cause stress to a cell so as to induce, activate or stimulate production of molecules that are not expressed by the cell in the normal or resting conditions. These factors include but are not limited to hormones, growth factors, cAMP inducers, such as forskolin, Ca++ flux inducing molecules, such as macrophage-derived colony stimulating factor, and other small organic or inorganic molecules or peptides, heat, pressure, radiation, genetic alterations and pathogens. Non-limiting examples of genetic alterations include genetic diseases, wherein production of proteins is altered due to a genetic defect, or tumors wherein genetic alterations have changed the normal expression pattern of cells. Pathogens may include virus particles, bacteria, fingi, and other cellular pathogens. The preferred inducing factors include one or more of the following: FSH, LH, TNF, IFNγ, PMA, LPS, cycloheximide and Indomethacin or mixtures thereof. In the most preferred embodiment, a mixture of all is used. For example, one can use FSH (0.1 nM), LH (0.1 μM), TNF (0.1 μg/ml), IFNγ (0.1 μg/ml), PMA (1 ng/ml), LPS (0.1 μg/ml), cycloheximide (50 μg/ml) and Indomethacin (1 μg/ml) for 1-3 hrs to induce an ovarian cell as explained more detail in the following Examples. Alternatively induction can be performed in two different steps with two different mixtures of stimulating factors as described in the following Examples. The stimulatory factors or mixture thereof can be added to cell culture medium. Alternatively, the factor or mixture of factors can be administered to a live animal in a carrier solution in a number of ways including subcutaneous, intraperitoneal, intravenous, and intramuscular administration. Preferably the factor or mixture of factors is administered intraperitoneally.

A first library nucleic acids are created from the stimulated cell source, which can be a cell or a mixture of cells or an organ or tissue or mixture thereof. A second library of nucleic acids are created from the same source that is not exposed to the stimulatory factors. The term “cell source” or “cell” or “tissue” or “organ”, which are used interchangeably in the present specification, means any organ, tissue or eukaryotic cell type or a mixture thereof. The organ is preferably of murine or human origin, but can be any other multicellular organism as well. Preferably the cell is a mammalian cell, most preferably a murine or a human cell. The cell can be any cell type including, but not limited to, epithelial, endothelial, neuronal, adipose, and reproductive cell, such as cumulus, ovarian or sertoli cell. The cell may be a cell line, a stem cell, or a primary cell isolated from any tissue including, but not limited to brain, liver, lung, gut, stomach, fat, muscle, testes, uterus, ovary, skin, endocrine organ and bone, etc.

The term “library of nucleic acids” comprises isolated nucleic acids cloned into a vector. The term “nucleic acid” or “set of nucleic acids” means isolated DNA, RNA and cDNA. Preferably the nucleic acids of the present invention are RNA and cDNA. Total RNA or mRNA from the source cells can be isolated from the stimulated cells or tissues using standard methods. The RNA can either be directly subjected to a subtractive hybridization or alternatively is first reverse transcribed to form cDNA. Standard methods for isolating RNA, mRNA and producing cDNA are set forth, for example, in Sambrook and Russel, MOLECULAR CLONING: A LABORATORY MANUAL, 3rd Ed., Cold Spring Harbor Laboratory Press, Cold Spring Harbor, N.Y. (2001), the entirety of which is herein incorporated by reference.

As used herein, the term “vector” refers to a nucleic acid molecule capable of carrying or transporting another nucleic acid to which it has been linked. The term “expression vector” includes plasmids, cosmids or phages capable of synthesizing the proteins encoded by the isolated nucleic acids carried by the vector. Preferred vectors are those capable of autonomous replication and/expression of nucleic acids to which they are linked. In the present specification, “plasmid” and “vector” are used interchangeably as the plasmid is the most commonly used form of vector. Moreover, the invention is intended to include such other forms of expression vectors which serve equivalent functions and which become known in the art subsequently hereto.

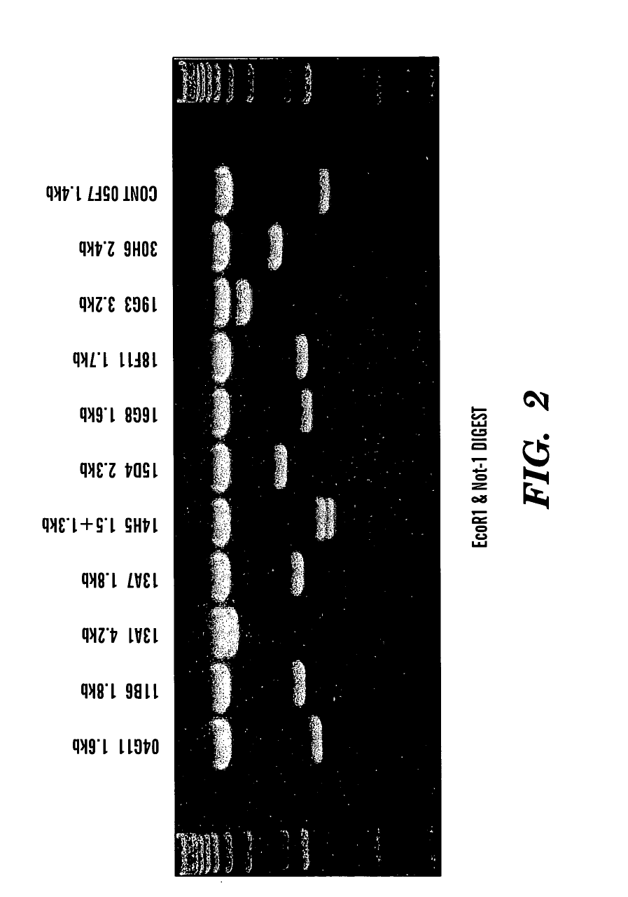

The nucleic acids of the first and second library are consequently subjected to subtractive hybridization between the RNA or cDNA from the unstimulated and stimulated cell or tissue. Preferably, the library contains at least about 1-2×10⁷ cDNA clones. The remaining nucleic acids are used to create a nucleic acid array on a filter or chip or on any suitable solid support wherein nucleic acids can be attached. Before subtractive hybridization, it is important to check whether the induction of cells was successful in the first step. This can be done by using, for example, reverse transcriptase polymerase chain reaction (RT-PCR) using primers that amplify a known inducible protein, such as a known cytokine, from the mixture of isolated nucleic acids. Methods for subtractive hybridization and consequent creation of subtractive cDNA library are routine and a detailed description of these methods can be found in, for example, Armen et al. Chapter 2 in Functional Genomics, eds. S. P. Hunt and F. J. Livesey, Oxford University Press., 2000, pp. 9-31, the entirety of which is herein incorporated by reference. The commercially available subtractive hybridization kits or reagents can be purchased, for example, from Amersham Pharmacia Biotech Inc., Piscataway, N.J., CLONTECH Laboratories Inc., Palo Alto, Calif., Invitrogen Corp., Carlsbad, Calif., Marin Biologic Laboratories Inc., Tiburon, CA and Vector Laboratories Inc., Burlingame, Calif. For example, FIG. 2 shows an example of clones after subtractive hybridization and creation of a cDNA library. The inserts have been digested using EcoR1 and Not-1 restriction enzymes.

The term “nucleic acid array” means a collection of nucleic acids that are attached to a solid support. The array is an orderly arrangement of isolated nucleic acids. It provides a medium for matching known and unknown DNA samples based on nucleic acid base-pairing rules and automating the process of identifying the unknown sequences which have higher expression when the source is induced. An array can be created on common assay systems such as microplates or standard blotting membranes, and can be created by hand or using robotics to deposit the sample. The term “nucleic acid array” relates to both macroarrays or microarrays, the difference being the size of the sample spots. Macroarrays contain sample spot sizes of about 300 microns or larger and can be easily imaged by existing gel and blot scanners. The sample spot sizes in microarray are typically less than 200 microns in diameter and these arrays usually contains thousands of spots. The method preferably uses microarrays. A nucleic acid microarray, or DNA or cDNA chip can be manufactured by high-speed robotics, for example, on glass or nylon substrates, for which probes created from the nucleic acids isolated from the stimulated library and the unstimulated library are used to determine complementary binding. This allows identification of nucleic acids that are differentially expressed in the stimulated and unstimulated source. For example, an array may be constructed using techniques described in U.S. Pat. No. 6,312,960, herein enclosed as a reference in its entirety. Alternatively, microarrays can be prepared by service providers, for example Incyte Genomics Inc., LifeArray service, Palo Alto, Calif. (www.incyte.com).

The nucleic acid array is hybridized with a first set of nucleic acids isolated from an stimulated source and a second set of nucleic acids isolated from an unstimulated source. The source may be the same as used for the creation of the libraries but it may also be a different source. The hybridization signals on the nucleic acid array that are more than about two times stronger after the hybridization of the first set compared to the hybridization of the second set are used to locate the clones in the first library which will be subjected to nucleic acid sequencing. The first and second set of nucleic acids are labeled using any detectable label including, but not limited to, radioactive labels such as P³³, P³², S³⁵, I¹²⁵ and the like, fluorophores such as fluorescein, luminescent labels, biotin, and digoxigenin. The detection is performed according to the type of label as known for the one skilled in the art. For example, detection of microarrays can be performed using a CCD-camera when the probe collection of isolated nucleic acids are labeled using a fluorescent dye.

Once the nucleic acids with at least about two times higher expression from the first, stimulated cell sources, as compared to the second, unstimulated cell sources, have been identified, a corresponding clone is picked from the original first library created from the stimulated cell source. The sequencing of the clones is performed using standard techniques from 5′ and/or 3′ ends of the clone to allow sequence comparison with existing sequences in the databases. Preferably sequencing is only partial sequencing. The nucleic acid sequencing is performed from both 5′ and 3′ ends of the clone to enable detection of a possible start codon, sequence encoding a signal peptide, and the poly-A signal. If these sequences are identified, the clone is likely to contain the coding sequence of a complete secreted protein and can be sequenced completely.

The 5′ and 3′ sequences are consequently subjected to a sequence comparison analysis using computer software such as BLAST [for BLAST programs, see Altschul, S. F. et al. (1990) “Basic local alignment search tool.” J. Mol. Biol. 215:403-410; Gish, W. & States, D. J. (1993) “Identification of protein coding regions by database similarity search.” Nature Genet. 3:266-272; Madden, T. L. et al., (1996) “Applications of network BLAST server” Meth. Enzymol. 266:131-141; Altschul, S. F. et al., (1997) “Gapped BLAST and PSI-BLAST: a new generation of protein database search programs.” Nucleic Acids Res. 25:3389-3402; Zhang, J. & Madden, T. L. (1997) “PowerBLAST: A new network BLAST application for interactive or automated sequence analysis and annotation.” Genome Res. 7:649-656 and for reviews se Altschul, S. F. & Gish, W. (1996) “Local alignment statistics.” Meth. Enzymol. 266:460-480; Wootton, J. C. & Federhen, S. (1996) “Analysis of compositionally biased regions in sequence databases.” Meth. Enzymol. 266:554-571; Altschul, S. F. et al., (1994) “Issues in searching molecular sequence databases.” Nature Genet. 6:119-129. Other BLAST related information is available at http://www.ncbi.nlm.nih.gov/BLAST/blast_references.html. The above mentioned references are herein incorporated in their entirety. If the nucleic acid sequence is less than about 50% homologous with any known sequence in the databases, it is considered a novel sequence. The homology is determined using standard setting of the sequence comparison.

If the nucleic acid clone in the first library contains only a partial protein encoding sequence, a complete clone can be fished out from any nucleic acid library such as YAC, PAC, P1, cosmid, plasmid or other library using standard cloning techniques such as PCR or hybridization as described in Sambrook and Russel, MOLECULAR CLONING: A LABORATORY MANUAL, 3rd Ed., Cold Spring Harbor Laboratory Press, Cold Spring Harbor, N.Y. (2001).

The partial sequencing of the clones is performed from 3′ and 5′ ends to allow sequence comparison with existing sequences. Sequencing both 3′ and 5′ ends of the clone also allows determination whether the clone is a full length clone or not. If the clone has a start codon and a sequence that encodes a signal peptide, the clone is likely a full length clone and can be directly sequenced from the library created from the first library of nucleic acids.

The common structure of signal peptides from various proteins is described as a positively charged n-region, followed by a hydrophobic h-region and a neutral but polar c-region. The (−3,−1)-rule states that the residues at positions −3 and −1 (relative to the cleavage site) must be small and neutral for cleavage to occur correctly. The signal peptides can be identified using computer software programs such as SIGFIND—Signal Peptide Prediction Server (Human), Version 2.04 DEC 12, 2001, by Synaptic Ltd. This software (SIGFIND2) predicts signal peptides at the start of protein sequences or searches open reading frames with a potential signal peptide coded in nucleotide sequences. The sig.pep. score along the sequence indicates the location and size of the signal-peptide. This score ranges from 0 (=no signal peptide) to 9 (=max. score for presence of a signal peptide). The range where this score drops from high to low indicates the approximate position of the cleavage site. Bidirectional recurrent neural networks (BRNNs) are used for prediction. It is trained on the human protein data used for the SIGNALP system described in H.Nielsen, et al., “Identification of prokaryotic and eukaryotic signal peptides and prediction of their cleavage sites” Protein Engineering, vol. 10 no. 1 pp. 1-6, 1997. The SIGNALP data is derived from A.Bairoch and B.Boeckmann, “The SWISS-PROT protein sequence data bank: current status”, Nucleic Acids Res. 22:3578-3580 (1994). Using the same fivefold cross-validation as SIGNALP, the 5 networks of SIGFIND2 (average correlation coefficient 0.99) perform better than SIGNALP (average correlation coefficient 0.96). The predictions of the 5 networks are combined into a jury decision. The BRNN algorithin is described in “Bidirectional Dynamics for Protein Secondary Structure Prediction” P. Baldi et al., in R Sun and L. Giles, editors, “Sequence Leaming: Paradigms, Algorithms, and Applications”, Springer Verlag, 2000.

The novel clones can subsequently be expressed either in a cell culture or in a transgenic animal model. After in vitro expression, the cell culture medium can be collected and the expressed molecules analyzed using a number of techniques. The typical approach used in assessing the number and identity of expressed proteins is a 2 dimensional (2D) gel electrophoresis and its extensions. The proteins are separated on the basis of size and charge. Typically, several thousands of proteins can be resolved on a single gel (O'Farrell, P. H., High resolution two-dimensional electrophoresis of proteins, J Biol Chem, 250, 4007, 1975).

Mass spectrometry (MS) is another method of analyzing proteins and can be used in conjunction with the 2D gels after proteolytic cleavage of proteins to quantitatively ascertain the mass associated with each fragment and eventually to identify the protein sequence.

Proteins of interest can be isolated using standard protein isolation techniques. The secreted proteins obtained using the present invention may be used to prepare so called protein chips. Such a chip comprises a substrate (e.g., a glass slide) and an array of proteins. The chips allow capture, separation and quantitative analysis of proteins directly on a chip. One method of performing a chip analysis is to integrate mass spectrometry (particularly, surface enhanced laser desorption/ionization (SELDI)) and biochip technology on a single chip. For example, ProteinChip™ (Ciphergen Biosystems, Inc.) uses various molecular substrates, including antibodies and receptors, having affinities for proteins of interest. The chips are made of aluminum, about three inches long and one centimeter wide, containing eight sites and a group of 12 can be processed as the equivalent of a 96-well format

Another protein chip assay, Protein 200 Plus LabChip kit, is available from Agilent Technologies, Inc.

A large-scale standardized methods for producing protein biochips can be obtained from, for example, Zyomyx Inc. (CA) and CombiMatrix Corp. (CA). These chips are covered with a multi-component organic thin film to reduce non-specific protein binding and a protein capture agent such as an antibody or a peptide to fish for specific proteins of interest. Methods for forming arrays of proteins and methods of use thereof are set forth in WO 00/04382 A1, the disclosure of which is incorporated herein by reference.

Protein chips or protein arrays can be used to screen for interaction of proteins with other proteins; (e.g., receptors), DNA, antibodies, cells, or small molecules before time consuming nucleic acid cloning and sequence analysis.

Clones which show interesting functions either in the cell cultures or in transgenic animals can consequently be sequenced using standard methods. Standard protocols for nucleic acid sequencing, cloning into expression vectors and creating transgenic animals are presented, for example, in Sambrook and Russel, MOLECULAR CLONING: A LABORATORY MANUAL, 3rd Ed., Cold Spring Harbor Laboratory Press, Cold Spring Harbor, N.Y. (2001).

Alternatively, the proteins can also be expressed and thereafter used to produce antibodies. Antibodies can be prepared by means well known in the art. The term “antibodies” is meant to include monoclonal antibodies, polyclonal antibodies and antibodies prepared by recombinant nucleic acid techniques that are selectively reactive with a desired antigen such as a polypeptide or protein or a mixture of polypeptides or proteins isolated using the method described above.

As used herein, the term “monoclonal antibody” refers to an antibody composition having a homogeneous antibody population. The term is not limited regarding the species or source of the antibody, nor is it intended to be limited by the manner in which it is made. The term encompasses whole immunoglobulins as well as fragments such as Fab, F(ab′)₂, Fv, and others which retain the antigen binding function of the antibody. Monoclonal antibodies of any mammalian species can be used in this invention. In practice, however, the antibodies will typically be of rat or murine origin because of the availability of rat or murine cell lines for use in making the required hybrid cell lines or hybridomas to produce monoclonal antibodies.

As used herein, the term “humanized antibodies” means that at least a portion of the framework regions of an immunoglobulin are derived from human immunoglobulin sequences.

As used herein, the term “single chain antibodies” refer to antibodies prepared by determining the binding domains (both heavy and light chains) of a binding antibody, and supplying a linking moiety which permits preservation of the binding function. This forms, in essence, a radically abbreviated antibody, having only that part of the variable domain necessary for binding to the antigen. Determination and construction of single chain antibodies are described in U.S. Pat. No. 4,946,778 to Ladner et al.

The term “selectively reactive” refers to those antibodies that react with one or more antigenic determinants of the desired antigen, such as a polypeptide or protein or a mixture of polypeptides or proteins isolated using the method described above, and do not react appreciably with other polypeptides. For example, in a competitive binding assay, less than 5% of the antibody would bind another protein, preferably less than 3%, still more preferably less than 2% and most preferably less than 1%. Antigenic determinants usually consist of chemically active surface groupings of molecules such as amino acids or sugar side chains and have specific three dimensional structural characteristics as well as specific charge characteristics. Antibodies can be used for diagnostic applications or for research purposes.

One method of generating such an antibody is by using hybridoma mRNA or splenic mRNA as a template for PCR amplification of such genes [Huse, et al., Science 246:1276 (1989)]. For example, antibodies can be derived from murine monoclonal hybridomas [Richardson J. H., et al., Proc Natl Acad Sci USA Vol. 92:3137-3141 (1995); Biocca S., et al., Biochem and Biophys Res Comm, 197:422-427 (1993) Mhashilkar, A. M., et al., EMBO J. 14:1542-1551 (1995)]. Other sources include transgenic mice that contain a human immunoglobulin locus instead of the corresponding mouse locus as well as stable hybridomas that secrete human antigen-specific antibodies. [Lonberg, N., et al., Nature 368:856-859 (1994); Green, L. L., et al., Nat Genet 7:13-21 (1994)]. Such transgenic animals provide another source of human antibody genes through either conventional hybridoma technology or in combination with phage display technology.

Once the protein immunogen is prepared, mice can be immunized typically twice intraperitoneally with approximately 50 micrograms of peptide or protein per mouse. Sera from such immunized mice can be tested for antibody activity by immunohistology or immunocytology on any host system expressing such polypeptide and by ELISA with the expressed polypeptide. For immunohistology, active antibodies of the present invention can be identified using a biotin-conjugated anti-mouse immunoglobulin followed by avidin-peroxidase and a chromogenic peroxidase substrate. Preparations of such reagents are commercially available; for example, from Zymad Corp., San Francisco, Calif. Mice whose sera contain detectable active antibodies according to the invention can be sacrificed three days later and their spleens removed for fusion and hybridoma production. Positive supernatants of such hybridomas can be identified using the assays described above and by, for example, Western blot analysis.

The present invention will now be illustrated by examples, which are not intended to be limiting in anyway, and make reference to the following figures.

### EXAMPLE 1

Rat ovary cells in culture were incubated for an hour with a following cocktail of stimulatory factors including FSH (0.1 nM), LH (0.1 μM), TNF (0.1 μg/ml), IFNγ (0.1 μg/ml), PMA (1 ng/ml), LPS (0.1 μg/ml), cycloheximide (50 μg/ml), and Indomethacin (1 μg/ml) for 1 hr. RNA from the cells was extracted using routine techniques. RNA was reversetranscribed into cDNA and the cDNAs were cloned into a vector. 10 novel clones were identified from 2000 partially sequenced clones. The clones were then digested using EcoR1 and Not-1 restriction enzymes. FIG. 2 shows the digests from 10 different clones of activated cDNA libraries and one from a non-activated, control cDNA library.

Primary libraries were constructed or directionally cloned, using at least 1 mg of total RNA with a SUPERSCRIPT™II RNase H⁻RT, ELECTROMAX™ DH10B cells and pCMV SPORT 6.1 vector.

Incorporation of radioactive label was used to evaluate first strand cDNA synthesis. The minimum specification was 15% incorporation (cDNA/mRNA). The libraries contained at least 3×10⁶ primary clones. Typical libraries had greater than 10⁷ clones.

23 clones were randomly picked and the average insert size was determined. The average insert size was at least 1.5-3 kb, typically average size was greater than 1.5 kb. In addition, the libraries typically had greater than 95% of vectors containing inserts.

### EXAMPLE 2

A mixture of FSH, TNF, IFN-γ is administered to a mouse in vivo. After 17 hours a second mixture containing FSH (0.1 nM), LH (0.1 μM), TNF (0.1 μg/ml), IFNγ (0.1 μg/ml), PMA (1 ng/ml), LPS (0.1 μg/ml), cycloheximide (50 μg/ml), and Indomethacin (1 μg/ml) was administered intraperitoneally (i.p.) to the same mouse in vivo. A control mouse receives no stimulatory factors but only PBS. Three hours after the administration of the second stimulatory mixture, both the stimulated and unstimulated mice are killed and RNA is extracted from their reproductive organs. A cDNA libraries are created from both control and induced mouse RNA samples and the libraries are subjected to subtractive hybridization. Subtracted transcripts are used to create a cDNA microarray which contains novel cDNA sequences. The microarray is hybridized using RNA obtained from the stimulated and unstimulated samples that are reverse transcribed to form cDNA and labeled with a different fluorescent dye (control is labeled with a different dye than the stimulated cDNA sample) from the mouse organs and the analysis was performed. The resulting stimulated genes, whose expression was at least about two times the expression of the unstimulated sample, are identified.

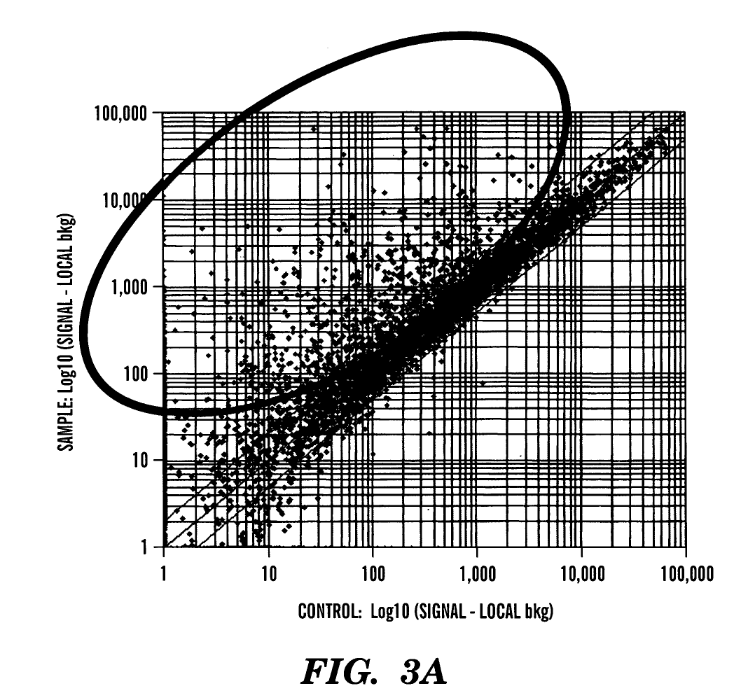

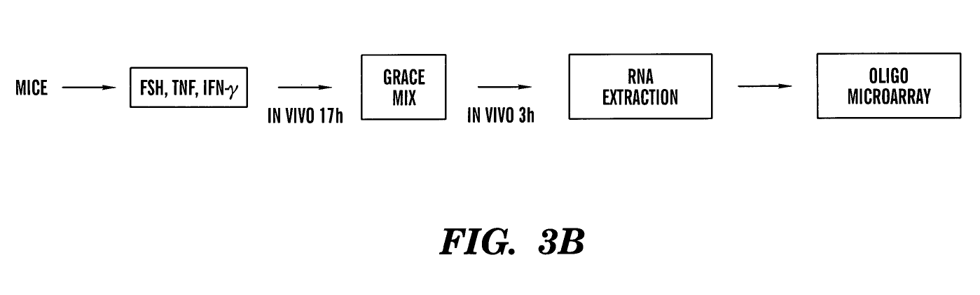

In FIG. 3, a commercial microarray (Incyte Genomics Inc., Palo Alto, Calif.) was hybridized with cDNA created from stimulated and unstimulated mouse reproductive organs as described above.

The preceding examples are to be evaluated as illustrative and are not intended to limit the scope of this invention.

All publications and patent applications cited in this specification are herein incorporated by reference as if each individual publication or patent application were specifically and individually indicated to be incorporated by reference. Although the foregoing invention has been described in some detail by way of illustration and an example for purposes of clarity of understanding, it will be readily apparent to those of ordinary skill in the art in light of the teachings of this invention that certain changes and modifications may be made thereto without departing from the spirit or scope of the appended claims.

1. A method of identifying a protein expressed in response to a stimulatory factor comprising the steps of:
a) exposing a first cell source to one or more stimulatory factors,
b) creating a first library of nucleic acids isolated from the stimulated first source,
c) creating a second library of nucleic acids from the first cell source not exposed to the stimulatory factors,
c) creating an array of nucleic acids by subjecting the first and second library to subtractive hybridization and creating an array of remaining nucleic acids,
d) taking a second cell source and exposing the second source to one or more stimulatory factors and isolating nucleic acids from the second source with and without stimulation, and
e) hybridizing the nucleic acids from the second source with and without stimulation to the array, wherein increased signal from the stimulated source indicates an expressed protein.

2. The method of claim 1 further comprising a step of picking a clone corresponding to the increased signal from the first library and sequencing the clone.

3. The method of claim 2 further comprising a step of subjecting the sequence of the clone to a sequence comparison software wherein a sequence that has less than about 50% homology with known sequences is a novel sequence.

4. The method of claim 3 further comprising a step of expressing the novel sequence.

5. The method of claim 1, wherein the nucleic acid is RNA or cDNA.

6. The method of claim 1, wherein the cell source is a reproductive cell.

7. The method of claim 3 wherein the reproductive cell is an ovarian cell.

8. The method of claim 1 wherein the stimulatory factor comprises one or more compounds selected from a group consisting of FSH, LH, TNF, IFNγ, PMA, LPS, cycloheximide and Indomethacin.

9. The method of claim 1 wherein the stimulatory factor is selected from a group comprising a pathogen, genetic defect, radiation, heat, a hormone, a growth factor, a cytokine, or mixture thereof.

10. The method of claim 1, wherein the cell source is an organ or a mixture of organs.

11. The method of claim 10 wherein the organ is a reproductive organ.

12. The method of claim 11 wherein the reproductive organ is an ovary.

13. A nucleic acid obtained by the method of claim 1.

14. A protein obtained by the method of claim 1.

15. The protein of claim 14, wherein the protein is a cytokine.

16. An array of nucleic acids obtained by the method of claim 1.

17. The method of claims 1 wherein the exposure of step (a) is performed in vitro.

18. The method of claim 1 wherein the exposure of step (a) is performed in vivo.

19. The method of claim 18 wherein the in vivo exposure is intraperitoneal.

20. The protein if claim 14, wherein the protein is an intracellular protein.

21. A method of producing an antibody against a protein expressed in response to a stimulatory factor comprising the steps of:
a) exposing a first cell source to one or more stimulatory factors,
b) creating a first library of nucleic acids isolated from the stimulated first source,
c) creating a second library of nucleic acids from the first cell source not exposed to the stimulatory factors,
c) creating an array of nucleic acids by subjecting the first and second library to subtractive hybridization and creating an array of remaining nucleic acids,
d) taking a second cell source and exposing the second source to one or more stimulatory factors and isolating nucleic acids from the second source with and without stimulation,
e) hybridizing the nucleic acids from the second source with and without stimulation to the array, wherein increased signal from the stimulated source indicates an expressed protein,
f) expressing the nucleic acids of step (e) to produce peptides, and
g) producing antibodies against the peptides.
